SEQUENCE LISTING (3 sequences)

SEQ ID NO: 1 - protein, 8 aa, Artificial Sequence
  synthetic peptide
SIINFEKL

SEQ ID NO: 2 - protein, 8 aa, Artificial Sequence
  Synthetic Peptide
SIINFEKL

SEQ ID NO: 3 - protein, 8 aa, Artificial Sequence
  Synthetic Peptide
SIINFEKL

(19) United States
(12) Patent Application Publication
(10) Pub. No.: US 2022/0133884 A1
(43) Pub. Date: May 5, 2022

(54) COMPOUNDS USEFUL AS ADJUVANTS

(71) Applicant: MILLENNIUM PHARMACEUTICALS, INC., Cambridge, MA (US)

(72) Inventor: Mithun KHATTAR, Roslindale, MA (US)

(21) Appl. No.: 17/434,112

(22) PCT Filed: Feb. 26, 2020
(86) PCT No.: PCT/US2020/019931

§ 371 Date: Aug. 26, 2021

Related U.S. Application Data
(60) Provisional application No. 62/810,777, filed on Feb. 26, 2019.

(51) Int. Cl. A61K 39/39; A61P 37/04

(52) U.S. Cl. CPC A61K 39/39; A61K 2039/55511; A61P 37/04

(57) ABSTRACT

The present disclosure provides heteroaryl compounds and pharmaceutically acceptable salts thereof useful as adjuvants and their use in pharmaceutical compositions such as vaccines. Further disclosed is the use of heteroaryl compounds and pharmaceutically acceptable salts thereof for stimulating an immune response in a subject.

### REFERENCE TO SEQUENCE LISTING SUBMITTED ELECTRONICALLY

The content of the electronically submitted sequence listing in ASCII text file (Name 3817_049PC01_SL_ST25; Size: 794 bytes; and Date of Creation: Feb. 25, 2020) filed with the application is incorporated herein by reference in its entirety.

### FIELD

The present disclosure provides heteroaryl compounds and pharmaceutically acceptable salts thereof useful as adjuvants and their use in pharmaceutical compositions such as vaccines. Further disclosed is the use of heteroaryl compounds and pharmaceutically acceptable salts thereof for stimulating an immune response in a subject.

### BACKGROUND

Vaccines are used to stimulate an immune response within an individual with the intent of providing protection against and/or treating a particular disease or condition. Some vaccines induce an immune response through the use of antigens. However, administering an antigen on its own rarely induces an adequate immune response. The use of an adjuvant along with the antigen can elicit an immune response that is faster or greater than that of the antigen alone and can often reduce the amount and dosing frequency of antigen that is required to stimulate a response. In addition, adjuvants may be used to direct the immune response to specific immunological pathways and to serve as a delivery vehicle for the antigen.

While numerous adjuvants are known, few have been approved for human use due to the adverse side effects that come with most adjuvant formulations. The currently approved adjuvants reflect a compromise between their activity and undesirable side-effects. Development of new adjuvants is often hindered by a particular adjuvant's effectiveness being linked only to a small number of antigens. In addition, reliable animal models are often not available which can lead to the failure of promising formulations to show efficacy in clinical trials.

While the most common adjuvants for human use today are aluminum hydroxide and aluminum phosphate, small molecule adjuvants offer improvements over these agents. As biologically active molecules, small molecules are amenable to the large number of drug-discovery techniques used to optimize their properties, such as rational drug design and computational approaches like QSAR. In principal, any property or characteristic can thus be designed in or out of the compounds, which allows the small molecule adjuvants to be tailored to specific biological functions, such as targeting specific cells or pathways to better address different diseases. There is, therefore, a need to provide new small molecule adjuvants that can be used as effective prophylaxis or in the treatment of various diseases and conditions, such as infectious diseases or cancer.

### SUMMARY

In a first aspect, the present disclosure provides a method for eliciting or enhancing an immune response in a subject in need thereof, the method comprising administering to the subject one or more antigens and a therapeutically effective amount of an adjuvant comprising a compound of Compound I-263a:

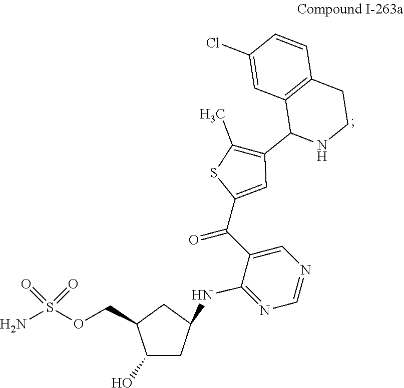 [SMILES: Cc1sc(C(=O)c2cncnc2N[C@@H]2C[C@H](COS(N)(=O)=O)[C@@H](O)C2)cc1C1NCCc2ccc(Cl)cc21]

or a pharmaceutically acceptable salt thereof.

In a first embodiment of the first aspect, the adjuvant is provided in a form admixed or co-formulated with the one or more antigens. In another embodiment, the adjuvant is formulated for parenteral administration. In another embodiment, the adjuvant is administered by a route selected from subcutaneous, intravenous, intradermal, and intramuscular administration.

In another embodiment of the first aspect, the adjuvant further comprises a TLR9 agonist. In another embodiment the TLR9 agonist is a CpG oligodeoxynucleotide.

In a second aspect, the present disclosure provides a method of activating the antigen-presenting function of antigen-presenting cells comprising administering, as an adjuvant with one or more different antigens, to a subject in need thereof, Compound I-263a:

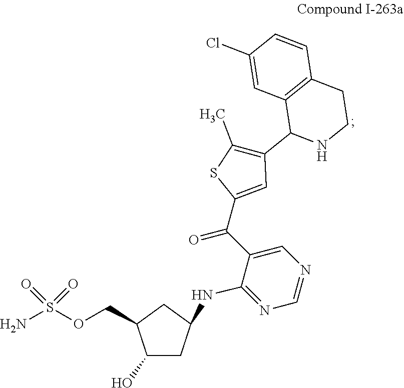 [SMILES: Cc1sc(C(=O)c2cncnc2N[C@@H]2C[C@H](COS(N)(=O)=O)[C@@H](O)C2)cc1C1NCCc2ccc(Cl)cc21]

or a pharmaceutically acceptable salt thereof.

In a first embodiment of the second aspect, the adjuvant is provided in a form admixed or co-formulated with the one or more antigens. In another embodiment, the adjuvant is formulated for parenteral administration. In another embodiment, the adjuvant is administered by a route selected from subcutaneous, intravenous, intradermal, and intramuscular administration.

In a third aspect, the present disclosure provides a method for stimulating an immune response in a subject in need thereof, the method comprising administering to the subject an immunostimulating effective amount of Compound I-263a:

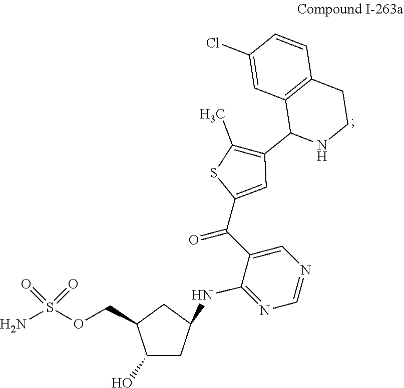 [SMILES: Cc1sc(C(=O)c2cncnc2N[C@@H]2C[C@H](COS(N)(=O)=O)[C@@H](O)C2)cc1C1NCCc2ccc(Cl)cc21]

or a pharmaceutically acceptable salt thereof.

In a first embodiment of the third aspect, the method further comprises administering a TLR9 agonist.

In a fourth aspect, the present disclosure provides a pharmaceutical composition, comprising Compound I-263a:

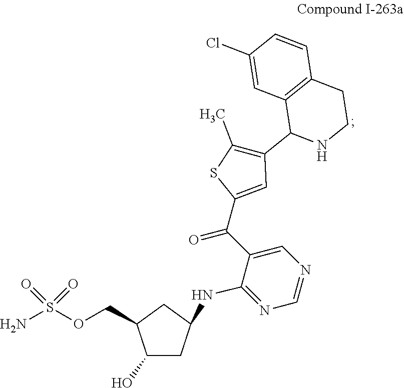 [SMILES: Cc1sc(C(=O)c2cncnc2N[C@@H]2C[C@H](COS(N)(=O)=O)[C@@H](O)C2)cc1C1NCCc2ccc(Cl)cc21]

or a pharmaceutically acceptable salt thereof; and one or more non-replicating antigens.

In a first embodiment of the fourth aspect, the pharmaceutical composition further comprises a TLR9 agonist.

### BRIEF DESCRIPTION OF THE FIGURES

FIG. 1A shows the increase of dendritic cells expressing the activation marker CD40 in brachial and inguinal lymph nodes 18 hours after subcutaneous injection of Compound I-263a. alone and in combination with CpG, relative to vehicle and CpG alone.

FIG. 1B shows the increase of dendritic cells expressing the activation marker CD86 in brachial and inguinal lymph nodes 18 hours after subcutaneous injection of Compound I-263a alone and in combination with CpG, relative to vehicle and CpG alone.

FIG. 2A shows the increase of T cells expressing the activation marker CD69 in brachial and inguinal lymph nodes 18 hours after subcutaneous injection of Compound I-263a alone and in combination with CpG, relative to vehicle and CpG alone.

FIG. 2B shows the increase of NK cells expressing the activation marker CD69 in brachial and inguinal lymph nodes 18 hours after subcutaneous injection of Compound I-263a alone and in combination with CpG, relative to vehicle and CpG alone.

FIG. 3 shows the increase of Kb-SIINFEKL tetramer positive CD8 T cells in brachial and inguinal lymph nodes 24 hours after treatment with OVA and either Compound I-263a alone or in combination with CpG, relative to OVA alone and the combination of OVA and CpG.

FIG. 4A shows the increase of SIINFEKL-specific CD8 T cells in spleens of mice treated with OVA and either Compound I-263a alone or in combination with CpG, relative to OVA alone and the combination of OVA and CpG 14 days after the initial treatment.

FIG. 4B shows the increase of CD8α+ dendritic cells loaded with the peptide SIINFEKL on H-2Kb 14 days after the initial treatment with OVA and either Compound I-263a alone or in combination with CpG, relative to OVA alone and the combination of OVA and CpG.

FIG. 5A shows the average growth of B16F10-OVA tumors implanted into female C57BL/6 mice following pre-treatment of mice with vehicle, Compound I-263a, or poly (I:C), or vaccination with OVA, with OVA+Compound I-263a, or with OVA+poly (I:C).

FIG. 5B shows the individual growth kinetics of B16F10-OVA tumors implanted into female C57BL/6 mice following treatment with vehicle, compared to treatment with I-263a, poly (I:C), OVA, OVA+Compound I-263a, or OVA+poly (I:C).

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entirety.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

### Pharmaceutical Compositions

In some embodiments the present disclosure provides an adjuvant comprising Compound I-263a:

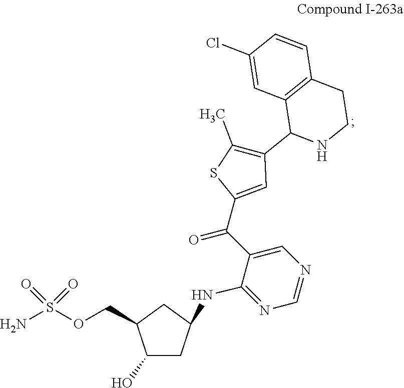 [SMILES: Cc1sc(C(=O)c2cncnc2N[C@@H]2C[C@H](COS(N)(=O)=O)[C@@H](O)C2)cc1C1NCCc2ccc(Cl)cc21]

or a pharmaceutically acceptable salt thereof. As used herein, the term “adjuvant” refers to a compound or compounds that, when used in combination with appropriate antigens, augment or otherwise alter or modify the resulting immune responses.

Compound I-263a, [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate, is an inhibitor of SUMO-activating enzyme, and is described in Example 201 of PCT publication number WO 2016/004136, which is hereby incorporated by reference in its entirety.

Compounds described herein may be in the form of a pharmaceutically acceptable salt. In some embodiments, such salts are derived from inorganic or organic acids or bases. For reviews of suitable salts, see, e.g., Berge et al., J. Pharm. Sci., 1977, 66, 1 19 and Remington: The Science and Practice of Pharmacy, 20th Ed., A. Gennaro (ed.), Lippincott Williams & Wilkins (2000).

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2 ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′ di(dehydro¬abietyl)-ethylene¬diamine), hydrobromide, hydrochloride, hydroxynaphthoate, iodide, isethionate (2 hydroxy¬ethanesulfonate), lactate, lactobionate, malate, maleate, mandelate, mesylate (methane¬sulfonate), methylbromide, methylnitrate, methylsulfate, mucate, napsylate (2-naphthalene¬sulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′ dibenzylethylenediamine), chloroprocaine, choline, diethanolamine, ethylenediamine, meglumine (N methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5 naphthalene¬disulfonate), oxalate, pectinate, persulfate, phenylethylbarbiturate, picrate, propionate, thiocyanate, tosylate and undecanoate; organic cations benethamine (N benzylphenethylamine), clemizole (1 p chloro¬benzyl-2 pyrrolildine-1′ ylmethylbenzimidazole), diethylamine, piperazine and tromethamine (tris(hydroxymethyl)aminomethane); and metallic cations barium and bismuth.

In other embodiments the adjuvant is provided in a form admixed with one or more antigens. As used herein, the term “antigen” means a compound or composition which, when introduced into an animal or a human in the appropriate context, provokes an immune response. This immune response may involve either antibody production, or the activation of specific immunologically-competent cells, or both. Examples of antigens include proteins, viruses, fungi, bacteria, toxins, chemicals, drugs, and foreign particles. “Non-replicating antigens” are antigens that do not replicate once inside the host.

In some embodiments the adjuvant further comprises a TLR9 agonist. TLR9 (toll-like receptor 9) is activated by unmethylated CpG-containing sequences, including those found in bacterial DNA or synthetic oligonucleotides (ODNs). Such unmethylated CpG containing sequences are present at high frequency in bacterial DNA, but are rare in mammalian DNA. Thus, unmethylated CpG sequences distinguish microbial DNA from mammalian DNA. A TLR9 agonist may be a preparation of microbial DNA, including, but not limited to, E. coli DNA, endotoxin free E. coli DNA, or endotoxin-free bacterial DNA from E. coli K12. In some embodiments, the TLR9 is a synthetic oligonucleotide containing unmethylated CpG motifs, also referred to herein as “a CpG oligodeoxynucleotide,” “CpGODNs,” “ODN,” or “CpG.” CpG ODNs are short, single stranded, DNA molecules that contain a cytosine (“C” nucleotide) followed by a guanine (“G” nucleotide). The “p” typically refers to the phosphodiester backbone of DNA. A TLR9 agonist of the present disclosure may include any of the at least three types of stimulatory ODNs have been described, type A, type B, and type C. CpG oligodeoxynucleotides may be produced by standard methods for chemical synthesis of polynucleotides or purchased commercially.

In some embodiments the pharmaceutical compositions described herein further comprise a pharmaceutically acceptable carrier. As used herein, the term “pharmaceutically acceptable carrier” includes any and all solvents, dispersion media, coatings, antibacterial, and antifungal agents, isotonic and absorption delaying agents, and the like, that are physiologically compatible. In one embodiment, the pharmaceutically acceptable carrier is suitable for subcutaneous, intravenous, intradermal, or intramuscular administration. Depending on the route of administration, the active compound may be coated in a material to protect the compound from natural conditions that may inactivate the compound.

Examples of suitable aqueous and non-aqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In other embodiments the pharmaceutical compositions described herein may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The compositions described herein may also contain adjuvants such as preservatives, wetting agents, emulsifying agents and dispersing agents. Prevention of the presence of microorganisms may be ensured both by sterilization procedures and by the inclusion of various antibacterial and antifungal agents, for example, paraben, chlorobutanol, phenol sorbic acid, and the like.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient(s) which can be combined with a carrier material to produce a single dosage form will vary depending upon the subject being treated, and the particular mode of administration. The amount of active ingredient(s) which can be combined with a carrier material to produce a single dosage form will generally be that amount of the composition which produces a therapeutic effect. Generally, out of one hundred percent, this amount will range from about 0.01 percent to about ninety-nine percent of active ingredient(s). In some embodiments the range is from about 0.1 percent to about 70 percent, and in other embodiments the range is from about 1 percent to about 30 percent of active ingredient(s) in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the disclosure are dictated by and directly dependent on (a) the unique characteristics of the active compound and the particular therapeutic effect to be achieved, and (b) the limitations inherent in the art of compounding such an active compound for the treatment of sensitivity in individuals.

For administration of Compound I-263a the dosage ranges from about 0.0001 to 100 mg/kg, and more usually 0.01 to 50 mg/kg, for instance about 1 mg/kg to about 25 mg/kg of the host body weight. For example, dosages can be 0.3 mg/kg body weight, 1 mg/kg body weight, 3 mg/kg body weight, 5 mg/kg body weight or 10 mg/kg body weight. An exemplary treatment regime entails administration once per day, once per week, once every two weeks, once every three weeks, once every four weeks, once a month, once every 3 months, or once every 3 to 6 months.

Actual dosage levels of the active ingredient(s) in the pharmaceutical compositions may be varied so as to obtain an amount of the active ingredient(s) which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, or salt thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective amount” is the amount of the adjuvant which, when given to a subject in combination with the antigen(s), results in a decrease in severity of disease symptoms, an increase in frequency and duration of disease symptom-free periods, or a prevention of impairment or disability due to the disease affliction. One of ordinary skill in the art would be able to determine such amounts based on such factors as the subject's size, the severity of the subject's symptoms, and the particular composition or route of administration selected.

An “immunostimulating amount” is the amount of an adjuvant which, when given to a subject, elicits or increases the magnitude or quantities of the reaction of the cells and fluids of the body to the presence of a substance that is not recognized as a constituent of the body itself. One of ordinary skill in the art would be able to determine such amounts based on such factors as the subject's size, the severity of the subject's symptoms, and the particular composition or route of administration selected.

A composition disclosed herein can be administered via one or more routes of administration using one or more of a variety of methods known in the art. As will be appreciated by the skilled artisan, the route and/or mode of administration will vary depending upon the desired results. In some embodiments the routes of administration for the compounds and compositions described herein include, but are not limited to, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In other embodiments, the composition can be administered via a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art.

### Method of Use of Compounds and Compositions

In some embodiments the present disclosure provides a method for eliciting or enhancing an immune response in a subject in need thereof, the method comprising administering to the subject one or more antigens and a therapeutically effective amount of an adjuvant comprising Compound I-263a:

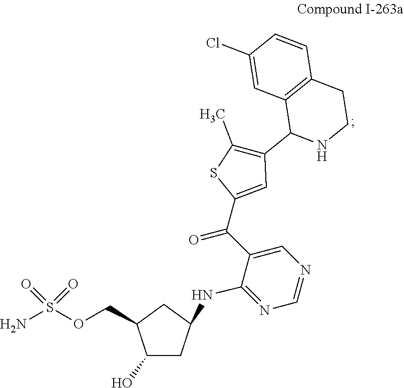 [SMILES: Cc1sc(C(=O)c2cncnc2N[C@@H]2C[C@H](COS(N)(=O)=O)[C@@H](O)C2)cc1C1NCCc2ccc(Cl)cc21]

or a pharmaceutically acceptable salt thereof. As used herein, “enhancing an immune response” means to increase the magnitude or quantities of the reaction of the cells and fluids of the body to the presence of a substance that is not recognized as a constituent of the body itself. The immune response can be an humoral response, which involves the transformation of B cells into plasma cells that produce and secrete antibodies to a specific antigen, and/or a cell-mediated response produced when sensitized T cells directly attack foreign antigens and secrete lymphokines that initiate the body's humoral immune response.

In some embodiments the present disclosure provides a method of activating the antigen-presenting function of antigen-presenting cells comprising administering, as an adjuvant with one or more different antigens, to a subject in need thereof, Compound I-263a, or a pharmaceutically acceptable salt thereof. As used herein, the term “antigen-presenting cell” refers to a cell capable of displaying, acquiring, or presenting at least one antigen or antigenic fragment on, or at, its cell surface. In general, an antigen-presenting cell can be any cell that aids the enhancement of an immune response against an antigen or antigenic composition.

The adjuvants and compositions described herein can be used in vaccines. As used herein, the term “vaccine” refers to a composition comprising one or more antigens that is administered, typically with an adjuvant, to an animal or human to produce an antigen-specific immune response, including, but not limited to, the production of antibodies, cytokines, and/or other cellular responses.

The combinations of adjuvants and antigens described herein can be useful for the treatment of cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and bloodborne tumors (hematologic malignancies). The term “cancer” encompasses diseases of skin, tissues, organs, bone, cartilage, blood, and vessels. The term “cancer” further encompasses primary and metastatic cancers.

Non-limiting examples of solid tumors that can be treated with the disclosed compositions include pancreatic cancer, bladder cancer (including invasive bladder cancer), colorectal cancer, thyroid cancer, gastric cancer, breast cancer (including metastatic breast cancer), prostate cancer (including androgen-dependent and androgen-independent prostate cancer), renal cancer (including, e.g., metastatic renal cell carcinoma), liver cancer (including, e.g. hepatocellular cancer and intrahepatic bile duct), lung and bronchus cancer (including non-small cell lung cancer (NSCLC), squamous lung cancer, brochioloalveolar carcinoma (BAC), adenocarcinoma of the lung, and small cell lung cancer (SCLC)), ovarian cancer (including, e.g., progressive epithelial or primary peritoneal cancer), cervical cancer, uterine cancer (including, e.g. uterine corpus and uterine cervix), endometrial cancer, gastric cancer, esophageal cancer, head and neck cancer (including, e.g., squamous cell carcinoma of the head and neck, nasopharyngeal caner, oral cavity and pharynx), melanoma, neuroendocrine cancer (including metastatic neuroendocrine tumors), brain cancer (including, e.g., glioma/glioblastoma, anaplastic oligodendroglioma, adult glioblastoma multiforme, and adult anaplastic astrocytoma), neuroendocrine (including metastatic neuroendocrine tumors), bone cancer, and soft tissue sarcoma.

Non-limiting examples of hematologic malignancies that can be treated with the disclosed compositions include acute myeloid leukemia (AML), chronic myelogenous leukemia (CIVIL) (including accelerated CIVIL and CML blast phase (CIVIL-BP)), acute lymphoblastic leukemia (ALL), chronic lymphocytic leukemia (CLL), Hodgkin's disease (HD), non-Hodgkin's lymphoma (NHL) (including follicular lymphoma and mantle cell lymphoma), B-cell lymphoma (including diffuse large B-cell lymphoma (DLBCL)), T-cell lymphoma, multiple myeloma (MM), amyloidosis, Waldenstrom's macroglobulinemia, myelodysplastic syndromes (MDS (including refractory anemia (RA), refractory anemia with ringed siderblasts (RARS), (refractory anemia with excess blasts (RAEB), and RAEB in transformation (RAEB-T)), small lymphocytic lymphoma (SLL), marginal zone lymphoma, smoldering multiple myeloma, and myeloproliferative syndromes.

In some embodiments, compositions of the present disclosure are suitable for the treatment of breast cancer, lung cancer, ovarian cancer, multiple myeloma, acute myeloid leukemia or acute lymphoblastic leukemia. In some embodiments, compositions of the present disclosure are suitable for the treatment of NHL. In some embodiments, compositions of the present disclosure are suitable for the treatment of indolent NHL. In some embodiments, compositions of the present disclosure are suitable for the treatment of follicular lymphoma, small lymphocytic lymphoma, mantle cell lymphoma or marginal zone lymphoma. In some embodiments, compositions of the present disclosure are suitable for the treatment of diffuse large B-cell lymphoma (DLBCL) or chronic lymphocytic lymphoma (CLL). In some embodiments, compositions of the present disclosure are suitable for the treatment of multiple myeloma. In some embodiments compositions of the present disclosure are suitable for the treatment of ALL, AML, or MDS.

In other embodiments, compositions of the present disclosure are suitable for the treatment of inflammatory, cardiovascular and neurodegenerative disorders including, but not limited to, allergies/anaphylaxis, acute and/or chronic inflammation, rheumatoid arthritis, autoimmunity disorders, thrombosis, hypertension, cardiac hypertrophy, heart failure, Huntington's disease and Alzheimer's disease.

In other embodiments, compositions of the present disclosure are suitable for the treatment of infectious diseases such as herpes simplex virus, HIV (including HIV-1 and HIV-2), feline immunodeficiency virus (Hy), cytomegalovirus, Varicella Zoster Virus, hepatitis, Epstein Barr Virus (EBV), respiratory syncytial virus (RSV), and human papilloma virus (HPV).

In other embodiments, compositions of the present disclosure are suitable for the treatment of bacterial infections such as those caused by an Actinobacterium (including, but not limited to, mycobacterium such as M. tuberculosis and M. laprae), Salmonella, Neisseria, Borrelia, Chlamydia, and Bordatella.

In other embodiments, compositions of the present disclosure are suitable for the treatment of fungal infections such as those caused by Aspergillus, Blastomyces, Coccidiodes, and Pneumocystis.

In other embodiments, compositions of the present disclosure are suitable for the treatment of parasitic infections such as those caused by protozoans (including, but not limited to, Plasmodium such as P. falciparum, P. vivax, P. malariae, and P. ovale), Acanthamoeba, Entamoeba histolytica, Angiostronglylus, Schistosoma mansonii, Schistosoma haematobium, Schistosoma japonicum, Schistosoma mekongi, Cryptospordium, Anclyostoma, Entamoeba coli, Entamoeba dispar, Entamoeba hartanni, Entamoeba polecki, Wucheria bancrofti, Giardia, Leishmania, Enterobius vermicularis, Ascaris lumbricoides, Trichuris trichiura, Necator americanus, Ancylostoma duodenale, Brugia malayi, Onchocerca volvulus, Dracanculus medinesis, Trichinella, spiralis, Strongyloides stercoralis, Opisthorchis sinesis, Paragonimus sp, Fasciola hepatica, Fasciola magna, Fasciola gigantica, Taenia saginata, and Taenia solium.

In other embodiments, the present disclosure provides a kit comprising an adjuvant as described herein in a first container, and one or more antigens in a second container, wherein the adjuvant composition is not in contact with the one or more antigens.

These and other embodiments of the present disclosure will become evident upon reference to the following examples which are offered by way of illustration and not by way of limitation.

### Examples

### Abbreviations

CTL Cytotoxic T lymphocyte
LN Lymph node
OVA Chicken ovalbumin protein

### General Analytical Methods:

Unless otherwise stated, immune cell profiling was performed using flow cytometry on a BD (Beckton Dickinson) LSRII Fortessa.

For detection of antigen-specific CD8 T cells (that recognize SIINFEKL (SEQ ID NO. 1), the class-I MHC epitope of OVA), MHC-I tetramers loaded with the cognate peptide (SIINFEKL) (SEQ ID NO. 1) and labeled with a fluorescent tag were used (MBL International, Woburn, Mass.). MHC class I tetramer reagents are generated primarily using the method developed by Altman et al., who demonstrated that tetramers of MHC class I/peptide complexes could be used as probes for detection and quantitation of antigen-specific CTL (Science 1996, 274: 94-96).

Compound I-263a, as described herein and used in the Examples below, can be synthesized according to the procedures recited in Example 201 in PCT publication number WO 2016/004136, which is hereby incorporated by reference in its entirety.

### General Experimental Conditions:

Vaccination with Chicken Ovalbumin protein (OVA): OVA is a key reference protein for vaccination experiments. Ovalbumin, the major protein constituent of chicken egg whites, is a glycoprotein that is sufficiently large and complex to be mildly immunogenic. Consequently, it is widely used as an antigen for immunization research. For an efficient in vivo cytotoxic T lymphocyte (CTL) response against OVA, it needs to be acquired and proteolytically cleaved by antigen presenting cells (APCs) into short antigenic peptides, and subsequently complexed with Class-I MHC molecules to be presented to CD8 T cells. This phenomenon is called antigen-cross presentation and among APCs, dendritic cells (DCs) are the most efficient at inducing cross-presentation of exogenous antigens and elicitation of cognate antigen-specific immune responses (Banchereau et al, Nature, 1998). Vaccine adjuvants can potentiate cross-presentation of exogenous antigens via activation of DCs and other APCs. In this study, high quality OVA protein with low endotoxin levels (EndoFit Ovalbumin, InvivoGen, San Diego, Calif.; Catalog number: Vac-pova-10) was used, which was dissolved in endotoxin-free water, appropriate for in vivo use.

### Test Articles:

The following test articles were used in the studies presented:

1. CpG: CpG ODNs (Oligo-di-nucleotide) are synthetic oligonucleotides that contain unmethylated CpG dinucleotides in particular sequence contexts (CpG motifs). These CpG motifs are present at a 20-fold greater frequency in bacterial DNA compared to mammalian DNA. CpG ODNs are recognized by toll-like receptor 9 (TLR9) leading to strong immunostimulatory effects. CpG is a clinically approved adjuvant used in Heplisav-B vaccine, and has demonstrated adjuvant activity in several preclinical studies. In this study, a vaccine grade formulation of CpG, prepared under strict aseptic endotoxin-free conditions (ODN2395, VacciGrade™, InvivoGen, San Diego, Calif.; Catalog number: vac-2395-1) was used. A stock solution of 1 mg/mL of CpG was prepared in sterile endotoxin-free water.

2. Compound I-263a: A 2.5 mg/mL or 1.5 mg/mL stock solution of Compound I-263a was formulated weekly in 20% HPβCD and administered either subcutaneously or intravenously (as indicated in respective studies) based on exact animal body weight on each day of treatment, using a dosing volume of 10 mL/kg body weight. Final doses received were 7.5 mg/kg.

### Methods and Results:

Immunizations with Compound I-263a, CpG, with or without the antigen OVA, were performed in mice to evaluate the effects of compounds on different immune cell subsets and their ability to present antigens, as well as prevent growth of tumors expressing OVA.

### Study 1: Effects on Innate Immune Cells:

The goal of this study was to investigate the in vivo effects of Compound I-263a alone or in combination with a TLR9 agonist (CpG) with respect to the recruitment and activation of innate immune cells at regional draining lymph nodes (LNs). On day 0 in the evening, eight to ten week old female Balb/c mice (Jackson Labs, Bar Harbor, Me.) were injected subcutaneously bilaterally close to the two brachial LNs (50 μL each side) with one of the formulations shown in Table 1 (n=5 mice per group). On day 1 (˜18 hours post injection) the mice were euthanized. The inflamed brachial LNs as well as the distal non-inflamed inguinal LNs were harvested from each mouse individually. Single cell suspensions were generated by homogenizing the LNs on a 70μ cell strainer using 3 mL syringe plungers, followed by wash with FACS staining buffer (BD Biosciences, Cat #554657). The cell pellet was re-suspended in 100 μL of FACS buffer for staining on a 96-well U-bottom tissue culture plate (Corning). Each sample was stained using the flow cytometry panel (see Table 2 below) of antibodies followed by acquisition and analysis on BD LSRII Fortessa. For positive controls, 1 drop of ultracomp beads (Invitrogen, Cat #01-2222-42) were stained with 1 μL of the respective antibodies.

TABLE 1
Treatment Groups

| | | Volume | Compound | |
|---|---|---|---|---|
| | Name | Injected | I-263a | CpG |
| Group 1 | Vehicle (20% HIPβCD + | 50 μL each side | 0 | 0 |
| | physiological water) | | | |
| Group 2 | Compound I-263a (7.5 | 50 μL each side | 150 ug | 0 |
| | mg/kg) | | | |
| Group 3 | CpG (2.5 mg/kg) | 50 μL each side | 0 | 50 μg |
| Group 4 | Compound I-263a + CpG | 50 μL each side | 150 μg | 50 μg |

TABLE 2
Flow Cytometry Antibody Panel for Study 1

| Antibody | Conjugate | Vendor | Cat No. | Clone: | Lot No. | Dilution |
|---|---|---|---|---|---|---|
| CD11b | BV510 | BD Biosciences | 562950 | M1/70 | 7117805 | 1:100 |
| CD11c | PE-CF594 | BD Biosciences | 562454 | HL3 | 7153950 | 1:100 |
| I-A/I-E | eFluor450 | eBioscience | 48-5321-82 | S/114.15.2 | 4329946 | 1:200 |
| CD40 | APC | BD Biosciences | 558695 | 42817 | 7103767 | 1:100 |
| CD80 | FITC | BD Biosciences | 553768 | 16-10A1 | 6275825 | 1:100 |
| CD86 | PE-Cy7 | eBioscience | 25-0862-82 | GL1 | 4305893 | 1:100 |
| F4/80 | BV711 | BD Biosciences | 565612 | T45-2342 | 7206718 | 1:200 |
| NKp46 | BV605 | Biolegend | 137619 | 29A1.4 | B238586 | 1:100 |
| CD69 | PE | Biolegend | 104508 | H1.2F3 | B233416 | 1:200 |
| CD19 | BUV395 | BD Biosciences | 563557 | 1D3 | 7125716 | 1:100 |
| CD3 | BUV737 | BD Biosciences | 564380 | 17A2 | 7221794 | 1:100 |
| Live dead | APC-Cy7 | eBioscience | L34975 | | 1875106 | 1:1000 |

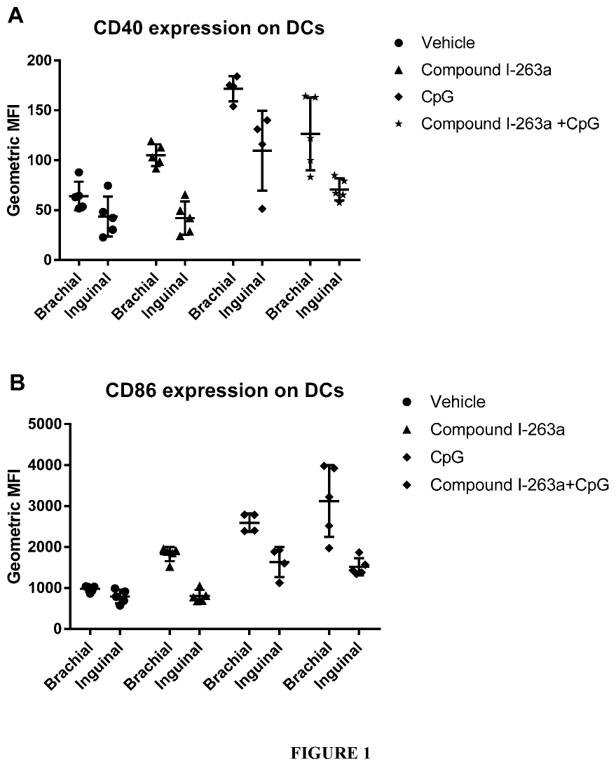

As shown in FIG. 1, subcutaneous injection of Compound I-263a at the brachial lymph nodes caused activation of dendritic cells, observed as increases in the expression of markers CD40 (FIG. 1a) and CD86 (FIG. 1b) on CD11c+ cells in the brachial LNs, but not in the distal inguinal LNs, relative to vehicle treated mice. Similar increases were also noted for CpG as well as for the combination of Compound I-263a+CpG.

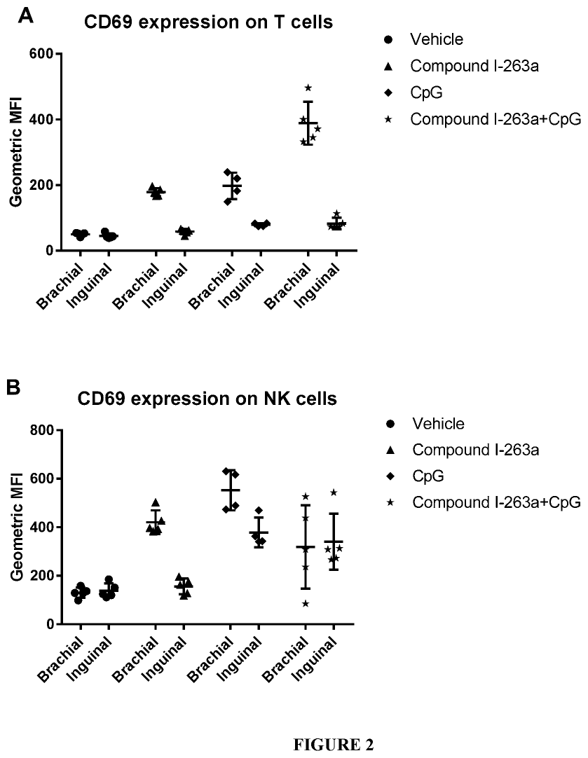

In addition, changes in the early lymphocyte activation marker CD69 were observed on T cells (FIG. 2a) and NK cells (FIG. 2b), upon administration of either Compound I-263a, CpG, or a combination of Compound I-263a+CpG in the brachial LNs. These results suggest that Compound I-263a is capable of activating both innate and adaptive immune cells in vivo, similar to CpG, a known TLR9 agonist and a potent vaccine adjuvant.

### Study 2: Effects on Antigen Presentation and T Cell Priming

The next concept investigated was whether enhanced activation of DCs translates to improved antigen presentation to T cells. Eight to ten week old female C57BL/6 mice (Jackson Labs, Bar Harbor, Me.) were injected with one of the formulations shown in Table 3 subcutaneously bilaterally close to the two brachial LNs (50 ul each side) on day 0 (n=10 mice per group). On Day 1 (˜18 hours post injection) five mice per group were euthanized. The inflamed brachial LNs as well as the distal non-inflamed inguinal LNs were harvested from each mouse individually. Single cell suspensions were generated by homogenizing the LNs on a 70 uM cell strainer using 3 mL syringe plungers, followed by wash with FACS staining buffer (BD Biosciences, Cat #554657). The cell pellet was re-suspended in 100 μL of FACS buffer for staining on a 96-well U-bottom tissue culture plate (Corning). Each sample was stained using the flow cytometry panel (see Table 3 below) of antibodies and Kb-SIINFEKL tetramer (SEQ ID NO. 3) (MBL International, Woburn, Mass.) followed by acquisition and analysis on BD LSRII Fortessa. For positive controls, 1 drop of ultracomp beads (Invitrogen, Cat #01-2222-42) were stained with 1 uL of the respective antibodies.

TABLE 3
Treatment groups for Study 2

| | | Volume | | Compound | |
|---|---|---|---|---|---|
| | Name | injected | OVA | I-263a | CpG |
| Group 1 | Vehicle (20% HPOCD + | 50 μL each | 100 μg | 0 | 0 |
| | physiological water) | side | | | |
| | + bug OVA | | | | |
| Group 2 | OVA + Compound I-263a | 50 μL each | 100 μg | 150 μL | 0 |
| | (7.5 mg/kg) | side | | | |
| Group 3 | OVA + CpG (2.5mg/kg) | 50 μL each | 100 μg | 0 | 50 μg |
| | | side | | | |
| Group 4 | OVA + Compound I-263a | 50 μL each | 100 μg | 150 μg | 50 μg |
| | + CpG | side | | | |

TABLE 4
Flow Cytometry Antibody Panel for Study 2

| Antibody | Conjugate | Vendor | Cat No. | Clone | Dilution |
|---|---|---|---|---|---|
| CD1 1 b | BV510 | BD | 562950 | M1/70 | 1:100 |
| | | Biosciences | | | |
| CD11c | PE-CF594 | BD | 562454 | HL3 | 1:100 |
| | | Biosciences | | | |
| CD4 | BV650 | Biolegend | 100546 | RM4-5 | 1:200 |
| CD8 | FITC | Invitrogen | MA517604 | KP1 | 1:100 |
| Kb- | APC | Biolegend | 141606 | 25-D1.16 | 1:100 |
| SIINFEKL | | | | | |
| (SEQ ID | | | | | |
| NO. 2) | | | | | |
| Kb- | PE | MBL | TB-5001-1 | | 1:50 |
| SIINFEKL | | | | | |
| Tetramer | | | | | |
| (SEQ ID | | | | | |
| NO. 3) | | | | | |
| F4/80 | BV711 | BD | 565612 | T45-2342 | 1:200 |
| | | Biosciences | | | |
| I-A/I-E | eF1uor450 | eBioscience | 48-5321-82 | S/114.15.2 | 1:200 |
| CD19 | BUV395 | BD | 563557 | 1D3 | 1:100 |
| | | Biosciences | | | |
| CD3 | BUV737 | BD | 564380 | 17A2 | 1:100 |
| | | Biosciences | | | |
| Live dead | APC-Cy7 | eBioscience | L34975 | | 1:1000 |

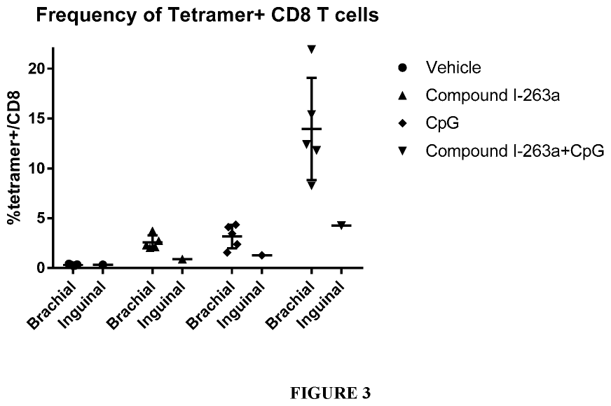

As shown in FIG. 3, an increase in the frequency of Kb-SIINFEKL tetramer (SEQ ID NO. 3) positive CD8 T cells was observed in the draining lymph nodes 18 hours after treatment with Compound I-263a+OVA, suggesting that Compound I-263a promotes cross-presentation of SIINFEKL (SEQ ID NO. 1), the class-I MHC epitope of OVA, to cognate antigen-specific CD8 T cells.

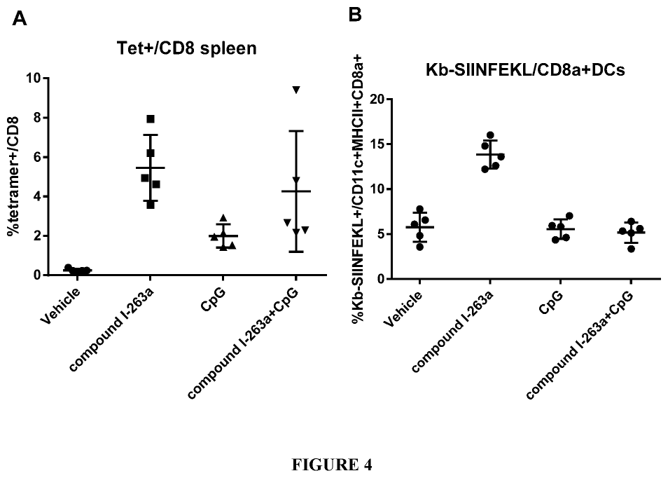

On day 7, the remaining 5 mice/group received a boosting dose of the formulations in Table 3, similar to those received on day 0. On day 14, the mice were euthanized and their spleens and LNs were harvested for testing the presence of tetramer+CD8 T cells as described above. Similar to day 0 results, SIINFEKL-specific CD8 T cells accumulated in the spleens of Compound I-263a+OVA treated mice, confirming that Compound I-263a promotes cross-presentation of extracellular antigens to CD8 T-cells (FIG. 4a). Notably, an increase in the frequency of CD8α+ DCs loaded with the peptide SIINFEKL (SEQ ID NO. 1) on H-2Kb (class-I MHC of C57BL/6 mice) was also observed, providing direct evidence for enhanced antigen-cross presentation (FIG. 4b).

These results demonstrate that Compound I-263a has the potential to act as a vaccine adjuvant by 1) activation of dendritic cells; and 2) promotion of antigen specific immune responses via cross-presentation of extracellular antigens by CD8α+ dendritic cells.

### Study 3: Effects on Tumor Growth

The goal of this study was to evaluate the prophylactic effects of OVA+Compound 1-263a vaccination in mice challenged with tumors expressing the OVA antigen (B16F10-OVA). The B16F10-OVA cell line was generated in-house by stable integration of the chicken ovalbumin gene into the Rosa26 locus. Eight to ten-week-old female C57BL/6 mice (Jackson Labs, Bar Harbor, Me.) were injected with one of the formulations shown in Table 5 subcutaneously (SC) near the nape. All mice were subsequently challenged on Day 0 with 0.3e6 B16F10-OVA tumor cells/mouse subcutaneously (n=16 mice/group, except group 5 (ovalbumin+Compound 1-263a) for which n=15). Tumor growth was measured every 2-3 days using digital calipers and tumor volume was calculated as: L×W×(W/2).

TABLE 5
Drug formulations administered to mice for Study 3

| | | | | | Number |
|---|---|---|---|---|---|
| Treatment | | | | | of |
| Group | Drug | Dosage | Regimen | Route | Animals |
| 1 | Vehicle | 20%HPbCD/ | Day - 14, | SC | 16 |
| | | 0.1ml (QWx2) | Day - 7 | | |
| 2 | Ovalbumin | 100 ug in 0.1 ml | Day - 14, | SC | 16 |
| | | physiological | Day - 7 | | |
| | | H₂0 (QWx2) | | | |
| 3 | Compound | 15 mg/kg in | Day - 14, | SC | 16 |
| | I-263a | 0.1m1 (BIWx2) | Day - 11 | | |
| | | | Day - 7, | | |
| | | | Day - 4 | | |
| 4 | Poly (I:C) | 50 ug in 0.1 ml | Day - 14, | SC | 16 |
| | | physiological | Day - 11 | | |
| | | H₂0 (BIWx2) | Day - 7, | | |
| | | | Day - 4 | | |
| 5 | Ovalbumin | 100 ug in 0.1 ml | Day - 14, | SC | 15 |
| | | physiological | Day - 7 | | |
| | Compound | H₂0 (QWx2) | Day - 14, | Sc | |
| | I-263a | 15 mg/kg | Day - 11 | | |
| | | in 0.1 ml (BIWx2) | Day - 7, | | |
| | | | Day - 4 | | |
| 6 | Ovalbumin | 100 ug in 0.1m1 | Day - 14, | Sc | 16 |
| | | physiological H₂0 | Day - 7 | | |
| | poly (I:C) | (QWx2)50 ug in | Day - 14, | Sc | |
| | | 0.1m1 physiological | Day - 11 | | |
| | | H₂0 (BIWx2) | Day - 7, | | |
| | | | Day - 4 | | |

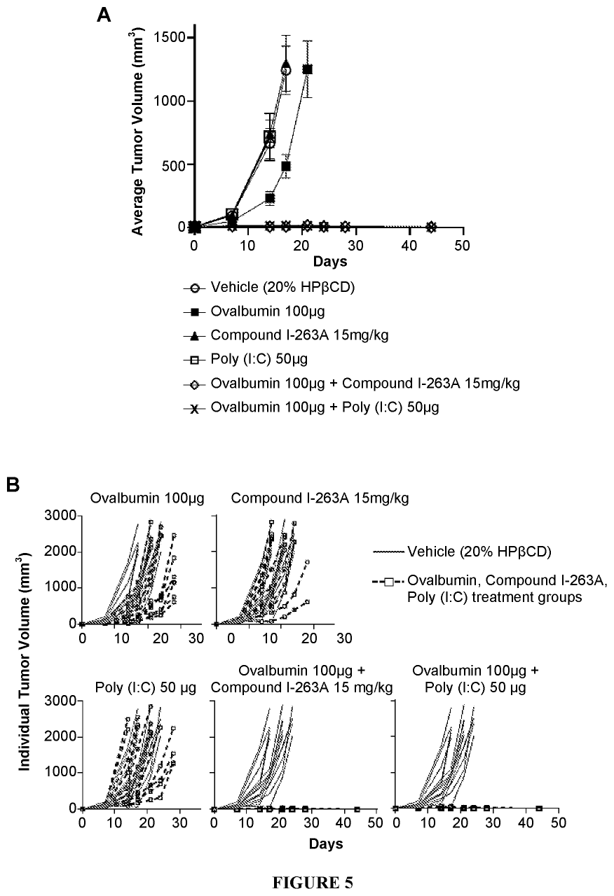

FIG. 5A shows average tumor volumes of each group up to the last day that the entire cohort within any given group was available for tumor size measurement, i.e. before mice were removed due to humane endpoints based on tumor size. FIG. 5B shows tumor volumes of individual mice within each group as marked. These figures demonstrate that vaccination with Compound I-263a+OVA protein completely prevented the growth of B16-F10 tumors expressing OVA, while either treatment alone did not confer tumor protection in mice. Similar results were observed for vaccination with OVA+Poly I:C (group 6), which is a TLR3 agonist and a widely used vaccine adjuvant. These results validate the adjuvant-like properties of Compound I-263a.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A method for eliciting or enhancing an immune response in a subject in need thereof, the method comprising administering to the subject one or more antigens and a therapeutically effective amount of an adjuvant comprising Compound I-263a: 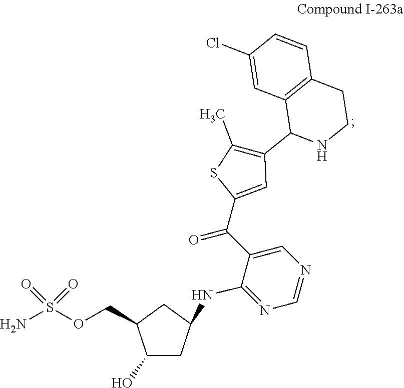 [SMILES: Cc1sc(C(=O)c2cncnc2N[C@@H]2C[C@H](COS(N)(=O)=O)[C@@H](O)C2)cc1C1NCCc2ccc(Cl)cc21]
or a pharmaceutically acceptable salt thereof.

2. The method of claim 1 wherein the adjuvant is provided in a form admixed or co-formulated with the one or more antigens.

3. The method of claim 1 wherein the adjuvant is formulated for parenteral administration.

4. The method of claim 3 wherein the adjuvant is administered by a route selected from subcutaneous, intravenous, intradermal, and intramuscular administration.

5. The method of claim 1 wherein the adjuvant further comprises a TLR9 agonist.

6. The method of claim 5 wherein the TLR9 agonist is a CpG oligodeoxynucleotide.

7. A method of activating the antigen-presenting function of antigen-presenting cells comprising administering, as an adjuvant with one or more different antigens, to a subject in need thereof, Compound I-263a. 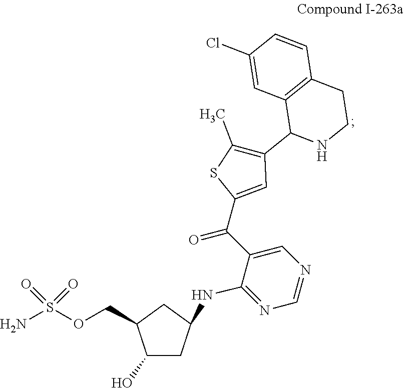 [SMILES: Cc1sc(C(=O)c2cncnc2N[C@@H]2C[C@H](COS(N)(=O)=O)[C@@H](O)C2)cc1C1NCCc2ccc(Cl)cc21]
or a pharmaceutically acceptable salt thereof.

8. The method of claim 7 wherein the adjuvant is provided in a form admixed or co-formulated with the one or more antigens.

9. The method of claim 7 wherein the adjuvant is formulated for parenteral administration.

10. The method of claim 9 wherein the adjuvant is administered by a route selected from subcutaneous, intravenous, intradermal, and intramuscular administration.

11. A method for stimulating an immune response in a subject in need thereof, the method comprising administering to the subject an immunostimulating effective amount of Compound 1-263a: 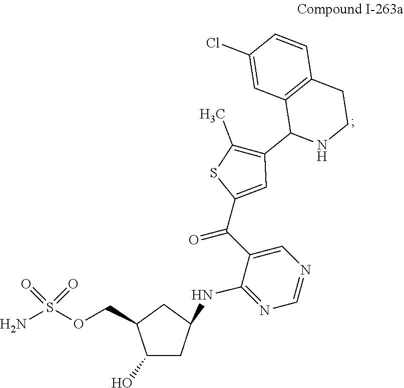 [SMILES: Cc1sc(C(=O)c2cncnc2N[C@@H]2C[C@H](COS(N)(=O)=O)[C@@H](O)C2)cc1C1NCCc2ccc(Cl)cc21]
or a pharmaceutically acceptable salt thereof.

12. The method of claim 11, further comprising administering a TLR9 agonist.

13. A pharmaceutical composition, comprising:
Compound 1-263a: 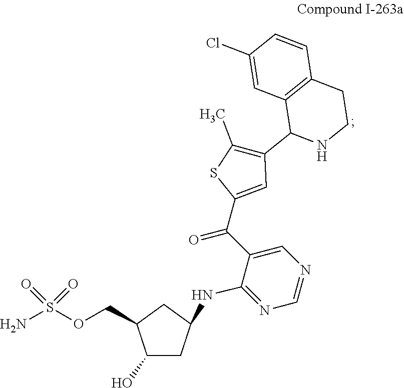 [SMILES: Cc1sc(C(=O)c2cncnc2N[C@@H]2C[C@H](COS(N)(=O)=O)[C@@H](O)C2)cc1C1NCCc2ccc(Cl)cc21]
or a pharmaceutically acceptable salt thereof; and
one or more non-replicating antigens.

14. The pharmaceutical composition of claim 13, further comprising a TLR9 agonist.
